(19) United States
(12) Patent Application Publication
(10) Pub. No.: US 2026/0007595 A1
(43) Pub. Date: Jan. 8, 2026

(54) EYE DROP FORMULATIONS CONTAINING CHONDROITIN SULFATE FOR RELIEVING OCULAR PAIN OR DISCOMFORT

(71) Applicant: SURFACE OPHTHALMICS, INC., Pleasanton, CA (US)

(72) Inventors: Richard L. Lindstrom, Wayzata, MN (US); Kamran Hosseini, Pleasanton, CA (US); Lyle Bowman, Pleasanton, CA (US)

(21) Appl. No.: 19/329,207

(22) Filed: Sep. 15, 2025

Related U.S. Application Data
(63) Continuation-in-part of application No. 18/654,457, filed on May 3, 2024, now Pat. No. 12,440,510.
Application No. 18/654,457 is a continuation-in-part of application No. 18/555,982, filed on Oct. 18, 2023, now Pat. No. 12,310,981.
(60) Provisional application No. 63/186,747, filed on May 10, 2021.

(51) Int. Cl. A61K 9/00; A61K 9/08; A61K 31/737; A61K 47/02; A61K 47/10; A61K 47/36

(52) U.S. Cl. CPC A61K 9/0048; A61K 9/08; A61K 31/737; A61K 47/02; A61K 47/10; A61K 47/36

(57) ABSTRACT

Composition and its use for use treating optical pain or discomfort, the composition being an eye dropper bottle or eye dropper dose vial containing an eye drop formulation having a viscosity about the same as water (e.g. 1-25 cps) and containing 0.1% to 2.5% wt/wt chondroitin sulfate. Optical pain or discomfort is treated by topically administering a therapeutically effective amount of the eye drops to a subject suffering from the optical pain or discomfort.

### CROSS-REFERENCE TO RELATED APPLICATIONS

This application is a continuation in part of U.S. patent application Ser. No. 18/654,457, filed May 3, 2024, which is a continuation in part of U.S. patent application Ser. No. 18/555,982, filed Oct. 18, 2023, now U.S. Pat. No. 12,310,981, which is a US national phase application of PCT/US2022/027177, filed May 1, 2022, which claims priority to U.S. provisional patent application No. 63/186,747, filed May 10, 2021.

This application is also a continuation in part of international patent application no. PCT/2025/024021, filed Apr. 10, 2025, which claims priority to U.S. patent application Ser. No. 18/654,457 filed 3 May 2024.

### TECHNICAL FIELD

The invention relates to eye drop formulations containing chondroitin sulfate and their use for treating ocular pain or discomfort.

### BACKGROUND OF THE INVENTION

Eye pain is generally characterized into two different categories: ocular pain, which occurs on the eye's surface; and orbital pain, which occurs within the eye. Ocular pain can be caused by allergy, conjunctivitis, irritation from contact lenses, corneal abrasion, burns, blepharitis, stye, dry eye disease, and other conditions.

Conventionally, ocular pain is treated with artificial tears. In more severe cases, eye drops incorporating a corticosteroid may be used. However, adverse and undesirable effects are commonly associated with steroid-based eye drops; and therefore, they are typically used sparingly. Among these include stinging, burning, itching or irritation of the eye, temporary cloudy vision, increased sensitivity to light, blurry vision, and allergic reactions.

Therefore, there remains a need for new therapeutic treatments to relieve ocular pain and discomfort with minimal adverse effects.

Chondroitin sulfate is one of the building blocks of cartilage and is believed to act as a protective coating for joints. In particular, it is believed that sulfate moieties of chondroitin sulfate interact with calcium ions in the bone to form a protective coating. WO 2006/058109 by Jafari et al. note that both chondroitin sulfate and glucosamine sulfate help build cartilage and can help delay the release of enzymes that breakdown cartilage. To this end, chondroitin sulfate has been used as a treatment to alleviate osteoarthritis pain and is often taken together with glucosamine sulfate.

In addition to its use in the treatment of osteoarthritis, WO 2006/058109 by Jafari et al. also describe a combination of chondroitin sulfate together with hyaluronic acid and glucosamine sulfate to form a triple polymer, viscoelastic agent to protect endothelial cells during cataract surgery. The term “viscoelastic” combines the words “viscous” and “elastic”. Viscous agents are resistant to flow, and elastic agents tend to return to their original shape after deformation. These features permit the Jafari et al. viscoelastic agent to be used during surgery to structurally maintain the corneal dome, maintain an anterior chamber depth (by filling the anterior chamber), and provide stability to the eye's anterior capsule surface. As such, Jafari et al. propose use of the triple polymer, viscoelastic agent at a surgical site during surgery, to protect the ocular tissue from trauma and therefore alleviate subsequent pain and inflammation associated with ocular surgery. Thus, while Jafari et al. disclose the use of chondroitin sulfate to act as a coating agent over the joints and to treat degradation of cartilage to alleviate osteoarthritis pain and the use of a viscoelastic agent to maintain optical structure during surgery to protect from trauma, Jafari et al. are silent as to the use of an eye drop formulation to treat ocular pain or discomfort after trauma has occurred.

U.S. Pat. No. 6,051,560 by Chang et al. found that adding chondroitin sulfate to sodium hyaluronate results in a synergistic viscosity due to hydrogen bonding between the molecules. Chang et al. disclose that administering this highly viscous composition before surgery protects corneal endothelial cells during intraocular lens (IOL) implantation, corneal transplantation, and other intraocular surgical operations. While Chang et al. provide a viscous solution for use before surgery, Chang et al. are silent as an eye drop formulation to treat optical pain or discomfort after trauma has occurred.

U.S. Pat. No. 4,486,416 by Soll et al. also disclose the use of chondroitin sulfate prior to ocular surgery. In particular, it was found that chondroitin sulfate can eliminate the electrical attractive force between an intraocular lens and the endothelial layer of the cornea and thus, dipping an intraocular lens in a viscous solution of about 10-20% chondroitin sulfate prior to applying the lens to the eye, was found to reduce trauma. While the viscous solution disclosed in Chang et al. provide a protective coating prior to surgery, Chang et al. are silent as to an eye drop formulation to treat optical pain or discomfort after the trauma has occurred.

U.S. Pat. No. 8,551,974 by Lindstrom found that a solution containing 2.5% chondroitin sulfate together with glycerol and dextran was an effective corneal pretreatment immediately before performing ophthalmic surgery. Consistent with prior uses of viscoelasitic agents, the composition formed a beneficial film over the cornea through which the surgery could be performed. While Lindstrom provides a film-like composition placed over the eye during surgery, Lindstrom is silent as to an eye drop formulation to treat optical pain or discomfort after the trauma has occurred.

In summary, chondroitin sulfate has been demonstrated to alleviate pain from osteoarthritis due to its ability to protectively coat joints and to reduce enzymatic degradation of cartilage. However, the mechanisms of osteoarthritis pain relief would not apply to the eye. Chondroitin sulfate has also been used in highly viscous formulations before and during ocular surgery. The viscous solution coats the eye to minimize trauma during ocular surgery. However, the technical approach of applying a viscous coating to protect from trauma would not apply to instances after surgery to treat pain or discomfort, nor instances where the pain and discomfort are not caused by surgery. Moreover, the use of viscous coatings would tend to cause blurred vision and would therefore be less desirable for frequent self-administration to treat optical pain.

Accordingly, there remains a need for new treatments to relieve ocular pain and discomfort that can be self-administered by a subject suffering from ocular pain or discomfort, whether the pain or discomfort occurs after surgery or not in response to surgery.

### SUMMARY OF THE INVENTION

The invention is directed to eye drop formulations containing a low concentration of chondroitin sulfate and their use for the topical treatment of ocular pain or discomfort. In contrast to previous approaches of using chondroitin sulfate in highly viscous solutions to provide a protective coating to protect cells or tissue during surgery, the technical approach of the invention is to provide chondroitin sulfate at a low concentration, which contributes very little to the viscosity of the solution. A low dose of chondroitin sulfate topically applied to the eye yielded surprising results when administered to subjects suffering from ocular pain. Moreover, this low dose of chondroitin sulfate is provided in an eye drop formulation having a viscosity about the same as water and therefore minimizes adverse effects consistent with viscous solutions, such as blurred vision, and minimizes adverse effects associated with steroid-based formulations.

The above benefits have been surprisingly achieved in one aspect of the invention, by a composition for use in the treatment of optical pain or discomfort, which includes an eye drop formulation having a viscosity about the same as water and containing chondroitin sulfate at a concentration of 0.1% to 2.5% wt/wt. The eye drop formulation is provided in an eye dropper bottle or eye dropper dose vial for topical application to the eye. This low concentration of chondroitin sulfate in a low viscosity formulation was found surprisingly effective at treating optical pain and can be self-administered by the subject in need of pain relief.

In some embodiments, the eye drop formulation also includes Dextran-70 and glycerol. In such embodiments, the concentrations of Dextran-70 and glycerol are such that they contribute little to the viscosity of the eye drop formulation so that the viscosity of the formulation remains about the same as water.

In preferred embodiments, the composition is free of hyaluronic acid and glucosamine sulfate.

While in some embodiments, the eye drop formulation contains a steroid, preferably the eye drop formulation is steroid free.

In a particularly preferred embodiment the eye drop formulation includes 0.1-2.5% wt/wt chondroitin sulfate, 0.1%-1.0% wt/wt dextran-70, 0.03% wt/wt potassium chloride, 0.20% wt/wt PLURONIC F-127, 0.1-2% wt/wt glycerol, 0.6% wt/wt sodium phosphate dibasic anhydrous, 0.1% wt/wt sodium phosphate monobasic dihydrous, and water.

In a related aspect of the invention, a composition for treating optical pain or discomfort is provided, where the composition includes an eye dropper bottle or an eye dropper dose vial containing an eye drop formulation, the eye drop formulation having a viscosity of 1 to 25 centipoise (cps) and chondroitin sulfate at a concentration of 0.1% to 2.5% wt/wt. In some embodiments the viscosity of the eye drop formulation is 1 to 12 cps. In some embodiments, the viscosity is 1 to 5 cps.

In some embodiments, the eye drop formulation also includes Dextran-70 and glycerol. In such embodiments, the concentrations of Dextran-70 and glycerol are such that they contribute little to the viscosity of the eye drop formulation so that the viscosity of the formulation remains between 1 to 25 cps, 1-12 cps, or 1-5 cps. While in some embodiments the eye drop formulation contains a steroid, in preferred embodiments the eye drop formulation is steroid free.

Again, an exemplary eye drop formulation includes 0.1-2.5% wt/wt chondroitin sulfate, 0.1%-1% wt/wt dextran-70, 0.03% wt/wt potassium chloride, 0.20% wt/wt PLURONIC F-127, 0.01-2% wt/wt glycerol, 0.6% wt/wt sodium phosphate dibasic anhydrous, 0.1% wt/wt sodium phosphate monobasic dihydrous, and water

In another related aspect of the invention, a method for treating ocular pain or discomfort is also provided. In particular, the eye drop formulation is topically administered to an eye of a subject suffering from ocular pain or discomfort. Among the benefits of the eye drop formulation is that it can be self-administered by the subject. Moreover, because the eye drop formulation has a viscosity about the same as water or about 1-25 cps (more preferably 1-5 cps), adverse effects associated with viscous solutions, such as blurred vision are minimal or nonexistent.

In some embodiments, the ocular pain or discomfort results from receiving a medical treatment, such as ocular surgery. Eye drop formulations containing chondroitin sulfate at low concentration are demonstrated herein to be surprisingly effective at reducing ocular pain or discomfort after undergoing ocular surgery. Thus, in some embodiments, topical administration of the eye drop formulation is used to treat pain or discomfort from an ocular surgery, which can be cataract surgery, refractive surgery or following intravitreal injections (IVI). In some embodiments, the eye drop formulation containing chondroitin sulfate is used to treat a subject suffering from surgical temporary ocular surgical discomfort syndrome (STODS).

In some embodiments the ocular pain or discomfort results from a medical condition, such as dry eye disease, contact lens induced ocular surface dry eye disease, preservative induced ocular surface disease, conjunctivitis, irritation from contact lenses, corneal abrasion, burns, blepharitis, or stye. Such uses include topically administering a therapeutically effective amount of the eye drop formulation containing 0.1% to 2.5% wt/wt chondroitin sulfate to the subject's eye that is suffering from the ocular pain or discomfort resulting from the medical condition.

In some embodiments, the optical pain or discomfort is from an allergy, such as an eye allergy. In such instances, use can include topically administering a therapeutically effective amount of the eye drop formulation containing 0.1% to 2.5% wt/wt chondroitin sulfate to the subject's eye that is suffering from the ocular pain or discomfort from the allergy.

In some embodiments, the optical pain or discomfort is from a corneal abrasion. In such instances, use can include topically administering a therapeutically effective amount of the eye drop formulation containing 0.1% to 2.5% wt/wt chondroitin sulfate to the subject's eye that is suffering from the ocular pain or discomfort from the corneal abrasion.

In some embodiments, the optical pain or discomfort is from wearing a contact lens. In such instances, use can include topically administering a therapeutically effective amount of the eye drop formulation containing 0.1% to 2.5% wt/wt chondroitin sulfate to the subject's eye that is suffering from the ocular pain or discomfort from wearing a contact lens.

In each of the embodiments, the eye drop formulation containing chondroitin sulfate has a viscosity about the same as water (e.g. 1-25 cps but more preferably 1-5 cps) and is administered topically to the eye. The eye drop formulation containing chondroitin sulfate can be administered over any time sufficient to reduce ocular pain or discomfort. In instances of ocular surgery, the eye drop formulation containing chondroitin sulfate can be administered over at least 7 days, optionally for 14 days or more after ocular surgery as a postop treatment regimen. In other instances, the eye drop formulation containing chondroitin sulfate can be self-administered whenever the subject feels pain or discomfort.

Chondroitin sulfate within the eye drop formulation can be at any suitable concentration from 0.1% to 2.5%, such as 0.1%, 0.15%, 0.2%, 0.25%, 0.3%, 0.35%, 0.4%, 0.45%, 0.5%, 0.55%, 0.6%, 0.65% 0.7%, 0.75% 0.8%, 0.85%, 0.9%, 0.95%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% wt/wt but the most preferred concentration is 0.1% to 0.25% wt/wt. Each of these concentrations of chondroitin sulfate contributes very little to the viscosity of the eye drop formulation, thereby avoiding significant changes in viscosity and minimizing instances of adverse effects such as blurred vision. Chondroitin sulfate can be derived from shark, bovine, or other sources.

### BRIEF DESCRIPTION OF DRAWINGS

FIG. 1 is a chart demonstrating percent pain clearance two weeks after cataract surgery, where the subjects received a topical eye drop formulation of either a combination of 0.2% wt/wt betamethasone sodium phosphate and 0.25% wt/wt chondroitin sulfate (left) or 0.25% wt/wt chondroitin sulfate (right).

### DETAILED DESCRIPTION OF THE INVENTION

As an introduction, the invention was surprisingly discovered while developing new steroid-based formulations for the treatment of ocular pain. Among these included a formulation where chondroitin sulfate was added as part of the delivery vehicle, given its known acceptance in formulations used during surgery. However, unlike its conventional use during surgery, the concentration was significantly reduced, and the solution itself had a viscosity close to water. This low concentration was used at least in part because chondroitin sulfate is known to affect viscosity. Chondroitin sulfate is a thickening agent. However, among the design parameters of the new steroid-based formulation was that it would have a low viscosity to 1) avoid overexposure to the steroid (due to adverse effects associated with steroids); 2) be configured as eye drops to be self-administered on the go by the subject; and 3) minimize blurred vision. Test formulations were developed to account for all three. Results during testing were surprising. Both the steroid formulation (+steroid/+chondroitin sulfate) and vehicle control (−steroid/+chondroitin sulfate) yielded unexpectedly high pain relief in the tested patient populations.

Accordingly, the invention surprisingly demonstrates the use of eye drop formulations having a viscosity about the same as water and containing chondroitin sulfate at a low concentration, suitable for self-administration for the treatment of ocular pain or discomfort. That is, it was surprisingly found that the self-administration of eye drops containing a low dosage of chondroitin sulfate as the active ingredient significantly reduced ocular pain. Pain was still further reduced when provided in combination with a steroid. It is believed that pairing chondroitin sulfate with a steroid will 1) offset or reduce adverse effects associated with steroid-based eye drops, and 2) permit steroids to be used at lower dose.

As introduced previously, chondroitin sulfate is a chemical normally found in cartilage around joints in the body. Commercially, it is both manufactured from animal sources (e.g. cow cartilage, shark cartilage) and produced chemically in the lab. Prior to cataract removal, chondroitin sulfate is typically injected into the eye to maintain a protective chamber during ocular surgery. While chondroitin sulfate is commonly used during surgical procedures in a viscous form, it is not conventionally prescribed during recovery. This disclosure newly documents the benefits of using a low dose of chondroitin sulfate as a topical treatment for the treatment of already occurring ocular pain or discomfort. This disclosure also newly documents the benefits of using chondroitin sulfate at low dose to treat pain or discomfort resulting from allergy, corneal abrasion, discomfort from wearing contact lenses, dry eye disease, ocular surgery induced dry eye disease, contact lens induced ocular surface dry eye disease, preservative induced ocular surface disease, surgical temporary ocular discomfort syndrome (STODS), and other ocular surface diseases and conditions that cause pain or discomfort. This disclosure also documents an eye drop formulation having a viscosity about the same as water and containing chondroitin sulfate at a concentration from 1-10% wt/wt, preferably 1-5% wt/wt, more preferably 0.1-2.5% wt/wt, more preferably 0.1-1.0% wt/wt and most preferably 0.1-0.25% wt/wt.

To assess the safety, tolerability and efficacy of various solutions for treating ocular pain, a multicenter, randomized, double-masked, parallel group study was conducted. In particular, the study included approximately 182 subjects who underwent uncomplicated unilateral cataract surgery (phacoemulsification or extracapsular extraction; surgery without the aid of a femto laser). The subjects were treated with either a pharmaceutical composition of 0.2% wt/wt betamethasone sodium phosphate and 0.25% wt/wt chondroitin sulfate or a pharmaceutical composition of 0.25% wt/wt chondroitin sulfate. In particular, one drop of study drug was self-instilled by the subject (or instilled by the subject's caregiver) BID as directed (once in the morning and once in the evening, preferably 8-12 hours apart) in the surgery eye (study eye) for 16 days: the day prior to cataract surgery, the day of cataract surgery (at least one hour prior to surgery and then once in the evening following surgery), and continued BID postoperatively for 14 days.

The betamethasone/chondroitin sulfate pharmaceutical composition contained betamethasone sodium phosphate (0.2% betamethasone sodium phosphate) sodium thiosulfate, sodium 0.25% wt/wt chondroitin sulfate, Dextran-70, edetate disodium, poloxamer 407, glycerin, potassium chloride, sodium phosphate dibasic anhydrous, sodium phosphate monobasic anhydrous, sodium hydroxide, hydrochloric acid, and sterile water for injection. The formulation was sterile and non-preserved.

The chondroitin sulfate composition contained sodium thiosulfate, 0.25% wt/wt sodium chondroitin sulfate, Dextran-70, edetate disodium, poloxamer 407, glycerin, potassium chloride, sodium phosphate dibasic anhydrous, sodium phosphate monobasic anhydrous, sodium hydroxide, hydrochloric acid, and sterile water for injection. The formulation was sterile and non-preserved.

Primary efficacy analysis was conducted using a last-observation-carried-forward (LOCF) method. The primary efficacy endpoint is the proportion of subjects with an ACC grade of 0 at Day 15. Anterior chamber cells were counted and graded according to TABLE 1.

TABLE 1
Grading and Counting of
Anterior Chamber Cells

| Grade | Cell Count |
|---|---|
| 0 | 0 |
| 1 | 1-10 |
| 2 | 11-20 |
| 3 | 21-50 |
| 4 | >50 |

The secondary efficacy endpoint is the proportion of subjects who achieve a pain score of 0 on the VAS (0 to 100 mm scale) at each post-surgical assessment. Differences in proportions between the active treatment (with 0.2% betamethasone sodium phosphate) and placebo (without steroid) were calculated for each time point, with a 95% confidence interval for the differences. The secondary efficacy analysis is the proportion of subjects in each treatment group with an ACF grade of 0 at each study visit.

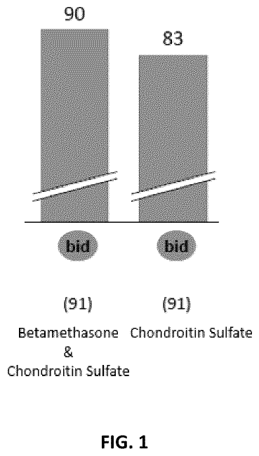

FIG. 1 is a chart of percent pain clearance at 2 weeks post-surgery. It was found that while the combined betamethasone/chondroitin sulfate composition was more efficacious than the chondroitin sulfate composition alone; the chondroitin sulfate composition, which is a steroid-free formulation performed surprisingly well even at low dose. The combined betamethasone/chondroitin sulfate treatment demonstrated 90% pain clearance and the chondroitin sulfate treatment demonstrated 83% pain clearance. This supports at least two specific uses for chondroitin sulfate. On the one hand, chondroitin sulfate can be combined with a corticosteroid in a pharmaceutical composition to provide a highly effective treatment against ocular pain, and on the other hand, chondroitin sulfate can also be provided in a steroid-free eye drop formulation for the treatment of ocular pain or discomfort.

One having ordinary skill in the art to which the invention belongs would appreciate that the above demonstrates new and surprising therapeutic uses for chondroitin sulfate. In particular, chondroitin sulfate was highly efficacious at clearing ocular pain (83%) and therefore provides a steroid-free alternative for treating ocular pain or discomfort. Moreover, the steroid-free use of chondroitin sulfate will have fewer adverse treatment effects than conventional steroid treatments. As such, the invention provides chondroitin sulfate itself as an active ingredient for the treatment, prevention or amelioration of ocular pain or discomfort alone or in combination with other therapeutics.

Still further, the chondroitin sulfate is formulated for use as eye drops, which can be self-administered by the subject suffering from ocular pain or discomfort. Eye drop formulations for self-administration have materially different properties compared to conventional chondroitin sulfate containing solutions used before and during surgery. In particular, the chondroitin sulfate-based eye drops incorporate a low concentration of chondroitin sulfate and have a significantly lower viscosity compared to configurations used prior to and during surgery. In particular, the eye drop formulations have a viscosity about the same as water (e.g. 1-25 cps).

An implication of varying the viscosity of a delivery solution is that it can materially affect the therapeutic activity of the active agent and its adverse effects. On the one hand, formulations having a higher viscosity tend to stay longer on the eye surface, which can extend exposure to the therapeutic agent. On the other hand, a viscous formulation can disturb the subject's vision and can be more difficult to self-administer. Here, it was surprisingly found that a low dose of chondroitin sulfate was highly effective at treating ocular pain even at low viscosity, which provided further surprising benefits because its administration no longer required a viscous or gel-like form. The result is a chondroitin sulfate based eye drop formulation having a viscosity close to water, which can therefore be self-administered without significant risk of visual disturbance.

The viscosity of preferred eye drop formulations disclosed herein are substantially lower than those conventionally used before and during surgery. This is accomplished at least in part by providing chondroitin sulfate at low concentrations (e.g. 0.1% to 2.5% wt/wt) since chondroitin sulfate is itself a known thickening agent. Moreover, maintaining a low viscosity can be achieved by choosing carriers and excipients that do not substantially affect the viscosity. As an example, hyaluronic acid and glucosamine sulfate, when used in combination with chondroitin sulfate, tend to thicken the formulation. As such, in preferred embodiments, the eye drop formulation is free of hyaluronic acid and glucosamine sulfate. However, compounds that affect the lubricious characteristics of the eye drop formulation but do not substantially affect the viscosity are preferred. As a nonlimiting example, a preferred eye drop formulation contains glycerol (e.g. 0.1%-2.0% wt/wt) and low concentration of Dextran-70 (e.g. 0.1% to 1%) to increase lubricity but at the same time, these amounts contribute minimally to the viscosity so it can remain close to that of water.

By having a viscosity “about the same as water” or “close to that of water,” it is meant that the viscosity of the formulation is from 1 cps or less, up to 25 cps at 20-25 degrees Celsius (“deg. C”). In comparison, a gel is typically about 500-20,000 cps. More preferably, the viscosity of the formulation is from 1 cps to 11 cps, which is still closer to water. In some embodiments, the viscosity is less than three-fold the viscosity of water. In still further embodiments, the viscosity is from half the viscosity of water to two-fold the viscosity of water. As will be evident in the passages that follow, variability in viscosity measurement can be due to the variability of instrument settings and testing parameters, shear rate, etc, and is therefore better provided as a range rather than an absolute value.

As further demonstration, the viscosity of the formulation in Example 5 was measured using a Brookfield viscometer Model RVDII with a CP-41 spindle at 24 deg. C. using 2 mL of fluid at shear rates of 8 to 200 sec⁻¹, and a Rheometric Scientific SR5 rheometer with 40 mm cone and plate at 25.0 deg. C. at shear rates (sec⁻¹) of 9.3 sec⁻¹ to 477 sec⁻¹. The same formulation on the SR5 gave an average of viscosity of 2.323 cps as compared to water 2.333 cps. The Brookfield viscometer measured the same formulation at 1.32 cps compared to water at 2.16 cps. A Brookfield viscometer DVT-4 with a #0 spindle at 24 deg. C. gave a viscosity of 1.25 cps at a shear rate 60 sec⁻¹. Formulations were made with additional chondroitin sulfate up to approximately 10% and resulted in Newtonian viscosities of 5 to 11 cps with variations due to source/process of chondroitin sulfate raw material. Additionally, even though we see viscosity differences from 1 cps to 12 cps by sensitive measurements, this viscosity difference is indistinguishable by the human eye. In measuring the viscosity of these thixotropic formulations (20 secc⁻¹ to 40 sec⁻¹) or the Newtonian viscosity.

Although it should be clear that a viscosity close to that of water is preferred, one having ordinary skill in the art to which the invention belongs would recognize that viscosity can be increased, if desired, by adding thickening agent(s) until the desired viscosity is achieved. However, increasing the viscosity may also increase adverse effects, such as blurred vision. Nonetheless, nonlimiting examples of thickening agents to increase the viscosity of the eye drop formulations can include carboxymethylcellulose (CMC), hydroxypropyl methylcellulose (HPMC), hyaluronic acid (HA), glucosamine sulfate (GS) or others known in the pharmaceutical industry. That is, while the invention documents chondroitin sulfate at low dose and at low viscosity is surprisingly effective at treating optical pain and discomfort, chondroitin sulfate can also be used at higher dose and at a viscosity substantially greater than water. Thus, chondroitin sulfate can be used at 0.1-2.5% wt/wt, 2.5%-10%, wt/wt, or more and at viscosities of 1-25 cps, 25-50 cps, 50-100 cps, 100-1,000 cps, or more than 1,000 cps; however as viscosities increase it my be desirable to increase the size of a dropper aperture to account for the passage of thicker fluids or provide a different topical application technique such as a gel coated substrate for placement on the surface of the eye to be treated.

One having ordinary skill in the art to which the invention belongs will appreciate that the eye drop formulations containing 0.1% to 2.5% chondroitin sulfate and others can also contain, preservatives (though not preferred), electrolytes, and buffering agents as desired.

The eye drop formulations are preferably contained in a suitable container for use in administering eye drops. These include eye dropper bottles (e.g. droptainer) and eye dropper dose vials as known in the art to which the invention belongs.

Turning now to the treatment of ocular pain or discomfort, the invention provides use of a composition, which itself is characterized by an eye drop formulation containing chondroitin sulfate at a concentration of 0.1% to 2.5% wt/wt in an eye dropper bottle or eye dropper dose vial, for treating ocular pain or discomfort, which includes topically administering to an eye of a subject suffering from ocular pain or discomfort, a therapeutically effective amount of the eye drop formulation containing 0.1 to 2.5% wt/wt chondroitin sulfate. Likewise, an exemplary method of treating ocular pain or discomfort can include topically administering to an eye of a subject suffering from a medical condition that results in ocular pain or discomfort, a therapeutically effective amount of the eye drop formulation containing 0.1% to 2.5% wt/wt chondroitin sulfate.

By “therapeutically effective amount” it is meant that the amount of administered chondroitin sulfate is effective to alleviate or ameliorate ocular pain or discomfort. The therapeutically effective amount may also alleviate inflammation; stinging, burning or scratchy sensation in the eyes; stringy mucus in or around the eyes; sensitivity to light; eye redness; sensation of having something in the eyes; difficulty wearing contact lenses; difficulty with nighttime driving; watery eyes; blurred vision and eye fatigue. It is envisioned that a therapeutically effective amount may vary depending on the disorder or condition and its severity, the age, weight, etc. of the subject to be treated. A therapeutically effective amount can be provided all at once in a single administration or can be fractional amounts that provide the effect in several administrations. The precise determination of what would be considered therapeutically effective may be based on factors individual to each subject, including their size, age, injury and or severity of disease being treated and amount of time since the injury occurred or the disease began. As general guidance one or a few drops of an eye drop formulation containing 0.1% to 2.5% of chondroitin sulfate should be effective for most uses, and a 0.1% to 0.25% wt/wt chondroitin sulfate solution formulated as eye drops tends to be most preferred. Thus, chondroitin sulfate can be provided at 0.1%, 0.15%, 0.2%, 0.25%, 0.3%, 0.35%, 0.4%, 0.45%, 0.5%, 0.55%, 0.6%, 0.65% 0.7%, 0.75% 0.8%, 0.85%, 0.9%, 0.95%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% wt/wt; each of these eye drop formulations has a viscosity about the same as water (e.g. 1-25 cps). One having ordinary skill in the art to which the invention belongs will be able to determine the therapeutically effective amount based on these conditions which are routine in the art.

In view of the above, the 0.1% to 2.5% chondroitin sulfate can be provided in an acceptable or suitable carrier, such as a diluent, buffer, or excipient, that is compatible with the chondroitin sulfate and any other ingredients forming the eye drop formulation and is suitable for topical administration to the eye as long as the viscosity remains about the same as water (e.g. 1-25 cps). A nonlimiting example is the formulation of eye drops for topical administration having chondroitin sulfate at a concentration of 0.1% to 2.5% wt/wt, or at a concentration of 0.1% to 1.0%, or 0.1% to 0.25% wt/wt. Thus, chondroitin sulfate can be provided at a concentration of 0.1%, 0.15%, 0.2%, 0.25%, 0.3%, 0.35%, 0.4%, 0.45%, 0.5%, 0.55%, 0.6%, 0.65% 0.7%, 0.75% 0.8%, 0.85%, 0.9%, 0.95%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or 2.5% wt/wt; each of these eye drop formulations has a viscosity about the same as water (e.g. 1-25 cps). When adding additional agents, the viscosity should be substantially maintained to remain about the same as water. For instance small amounts of dextran-70 (e.g. 0.1-1% wt/wt) and glycerol (e.g. 0.1-2% wt/wt) can be added.

In some embodiments, the eye drops consist essentially of chondroitin sulfate. By “consist essentially of” it is meant that the eye drop formulation lacks further active ingredients that materially affect the treatment, prevention or amelioration of symptoms associated with the condition to be treated, namely, ocular pain or discomfort. That is, while chondroitin sulfate can be used with a corticosteroid, it can also be used without a corticosteroid due to its efficacy of clearing ocular pain. Reagents such as those commonly associated with the preparation of eye drops, such as excipients, diluents, buffers, electrolytes, preservatives, and lubricants are not excluded by the term “consisting essentially of” as they do not themselves materially treat the underlying medical condition or symptom. An exemplary eye drop formulation that is steroid-free and/or “consists essentially of” chondroitin sulfate includes a combination of sodium thiosulfate, sodium chondroitin sulfate, Dextran-70, edetate disodium, poloxamer 407, glycerin, potassium chloride, sodium phosphate dibasic anhydrous, sodium phosphate monobasic anhydrous, sodium hydroxide, hydrochloric acid, and sterile water. Such ingredients can be provided in varying amounts as long as the viscosity of the eye drop formulation is about the same as water (e.g. 1-25 cps). Examples 1, 2, and 5 provide additional eye drop formulations that “consist essentially of” chondroitin sulfate and have a viscosity about the same as water.

As demonstrated herein, topical administration of eye drops containing chondroitin sulfate as a sole therapeutic agent at a low dose significantly relieved ocular pain after undergoing ocular surgery and is therefore demonstrated useful for treating various surgery related conditions such as surgical temporary ocular discomfort syndrome (STODS). STODS can occur following any ocular surgery or procedure that disrupts the ocular surface, such as refractive surgery, cataract surgery, and intravitreal injections (IVI). Without being bound by theory, it is believed that the chondroitin sulfate based eye drop formulation reduces cellular oxidated stress and edema to achieve cell membrane stabilization, enhances ocular surface lubrication and prolongation of contact time, and preserves ocular surface homeostasis by proactively addressing tear film hyperosmolarity.

The skilled artisan will appreciate that the eye drop formulations containing chondroitin sulfate can also be used to treat dry eye disease more broadly. Dry eye disease is frequently caused by decreased tear production. Decreased tear production can occur as a result of age, hormonal changes, various autoimmune diseases, and other factors, and may also be a side effect of certain medications, such as beta-blockers, antidepressants, antihistamines, etc.

Dry eye disease can also occur after ocular surgery. Manipulation and contact between instruments, medications, surgical tools, and the cornea can cause microscopic damage to the eye, thereby resulting in temporary disturbance to the ocular surface, which if left untreated can develop into a chronic condition. For patients with pre-existing dry eye, ocular surgery can exacerbate existing symptoms.

Eye drop formulations containing 0.1% to 2.5% wt/wt chondroitin sulfate can also be used to treat contact lens discomfort or contact lens induced ocular surface dry eye disease. Contact lens discomfort is often associated with dry eyes. A contact lens divides the tear film into two layers: the pre and post-lens tear film. Bifurcating the film into two layers increases friction between the contact lens and ocular surface, which can cause dry eye disease. Accordingly, also provided are 1) a method of treating ocular pain or discomfort from contact lens wear, which includes topically administering a therapeutically effective amount of an eye drop formulation containing 0.1% to 2.5% wt/wt chondroitin sulfate to an eye of a subject suffering from pain or discomfort from wearing contact lenses; and 2) a method of treating an ocular surface disease, which includes topically administering a therapeutically effective amount of an eye drop formulation containing 0.1% to 2.5% wt/wt chondroitin sulfate to an eye of a subject suffering from an ocular surface disease. In furtherance of this, in some embodiments ocular surface disease is a dry eye disease, such as contact lens induced ocular surface dry eye disease. In each, preferably the eye drop formulation has a viscosity that is about the same as water (e.g. 1-25 cps).

The eye drop formulations containing 0.1% to 2.5% wt/wt chondroitin sulfate can also be used to treat other ocular surface diseases. For example, glaucoma medications can be associated with toxicities to the ocular surface. Common therapies for glaucoma treatment include the use of prostaglandin analogs, beta-adrenergic antagonists, alpha-adrenergic agonists, and carbonic anhydrase inhibitors. Due to either the added preservative or the active ingredient of the medication itself, these topical treatments for glaucoma can cause or worsen ocular surface disease. Moreover, preservatives such as benzalkonium chlorides have widespread applications, including use in eye drops. Such preservatives can cause dry eye disease and are therefore preferably absent from the eye drop formulations. Accordingly, an eye drop formulation containing 0.1% to 2.5% wt/wt chondroitin sulfate can be used to treat ocular surface disease induced by preservatives or other active ingredients, such as those used in the treatment of glaucoma. As such, methods for treating ocular surface disease can include topically administering a therapeutically effective amount of an eye drop formulation containing 0.1% to 2.5% wt/wt chondroitin sulfate to an eye of a subject suffering from preservative induced ocular surface disease; and another exemplary embodiment includes topically administering a therapeutically effective amount of an eye drop formulation containing 0.1% to 2.5% wt/wt chondroitin sulfate to an eye of a subject suffering from glaucoma. Preferably, the viscosity is about the same as water to avoid blurred vision or other adverse effects associated with viscous solutions.

Eye drop formulations containing 0.1% to 2.5% wt/wt chondroitin sulfate can also be used to treat eye pain or discomfort from allergies, such as eye allergies. Eye allergy pain is caused by an inflammatory response triggered when the eyes come into contact with allergens like pollen, dust mites, pet dander, mold, or smoke.

Inflammation and irritation can cause blood vessel dilation (leading to redness), increased tear production, swelling (edema) of tissue, and/or stimulation of nerve endings resulting in pain, itching or burning sensations. Treatment of eye pain or discomfort from eye allergy can include topically administering to an eye of the subject suffering from the eye pain a therapeutically acceptable amount of an eye drop formulation containing 0.1% to 2.5% wt/wt chondroitin sulfate. In each of these, the viscosity is about the same as water (e.g. 1-25 cps). Without being bound by theory it is believed that chondroitin sulfate reduces edema and itchiness when used to treat allergy.

Though nonlimiting, this disclosure documents that topical administration of chondroitin sulfate at low dose, twice daily (BID) for two weeks, from the day of receiving cataract surgery, cleared 83% of ocular pain. As such, topical administration of eye drops containing chondroitin sulfate for less than two weeks after receiving ocular surgery will also clear pain; however, pain clearance might be below 83% in some subjects. Accordingly, within the scope of the disclosure, an eye drop formulation containing chondroitin sulfate is preferably topically administered for at least 1 day, 2 days, 3 days, 4 days, 5 days, 6 days, 7 days, 8 days, 9 days, 10 days, 11 days, 13 days, 14 days or more. While chondroitin sulfate can reduce pain within the first day, it has also been found that long term use is also effective. In some instances, eye drops containing chondroitin sulfate can be administered when the subject feels pain or discomfort. The artisan will also appreciate that while chondroitin sulfate is preferably administered topically via eye drops twice daily (BID), it can also be administered once daily, three times daily (TID), four times daily (QID) or more.

While the eye drop formulations containing chondroitin sulfate have been found highly efficacious at treating ocular pain, in some instances it may be beneficial to combine the chondroitin sulfate with a steroid, especially in instances of significant inflammation. For example, significant inflammation may occur after ocular surgery and thus a subject that has undergone ocular surgery may be provided postop with a pharmaceutical composition including chondroitin sulfate and a corticosteroid, preferably betamethasone sodium phosphate (e.g. 0.2% betamethasone sodium phosphate). In such compositions, the active ingredients (e.g. chondroitin sulfate and betamethasone sodium phosphate) are provided in a therapeutically effective amount. In furtherance of the above, the chondroitin sulfate was also administered topically to the eye with the corticosteroid betamethasone sodium phosphate during clinical testing. This combined betamethasone/chondroitin sulfate composition was administered twice daily (BID) for two weeks, from the day of receiving cataract surgery. The combined betamethasone/chondroitin sulfate composition cleared 90% of pain and 56% of inflammation. As such, the addition of a corticosteroid, preferably betamethasone sodium phosphate, is also encompassed by the methods and compositions described within this document.

For completeness, topical administration of chondroitin sulfate and a corticosteroid is preferably accomplished by way of administering both chondroitin sulfate and the corticosteroid in a same eye drop formulation; however, it is also envisioned that one composition could contain chondroitin sulfate in a steroid-free formulation and another composition could contain a corticosteroid in a chondroitin sulfate-free formulation provided separately. Though nonlimiting, an exemplary treatment regimen can include administering chondroitin sulfate and the corticosteroid for at least 1 day, 2 days, 3 days, 4 days, 5 days, 6 days, 7 days, 8 days, 9 days, 10 days, 11 days, 13 days, 14 days or more. The artisan will also appreciate that while the composition(s) is preferably administered topically via eye drops twice daily (BID), it can also be administered once daily, three times daily (TID), four times daily (QID) or more.

In still further treatment regimens, the subject is provided with both a steroid-free chondroitin sulfate formulation and a composition that includes the steroid. This approach may be preferred in instances where the steroid containing composition is to be used sparingly, such as in instances of severe inflammation or discomfort, while the formulation that is steroid-free is used more regularly for pain maintenance.

### EXAMPLES

### Example 1

### Preparing Eye Drop Formulation No. 1

Eye drop formulations were prepared as described below. The following products were used in the amounts specified in TABLE 2:

TABLE 2
Pharmaceutical Composition No. 1

| Amount | Product |
|---|---|
| 0.25 g | chondroitin sulfate (bovine) |
| 0.25 g | powdered dextran-70 |
| 0.1 g | edetate disodium powder |
| 0.03 g | potassium chloride |
| 0.20 g | PLURONIC ® F-127 |
| 0.2 g | glycerol |
| 0.9 g | sodium phosphate dibasic anhydrous |
| 0.18 g | sodium phosphate monobasic anhydrous |
| 100 mL | sterile injectable water |

Chondroitin sulfate, dextran, potassium phosphate, dibasic and monobasic sodium phosphate, potassium chloride, EDTA, and PLURONIC® F-127 were combined with about 90% of water and stirred until completely dissolved followed by adding glycerol with continued stirring. The pH of the solution was then adjusted to about 7.0-7.4 using sodium hydroxide solution. The solution was then filtered through a 0.2 micron filter into a sterile droptainer and sealed for subsequent topical delivery to the eye as eye drops. The viscosity of this solution is about the same as water.

### Example 2

### Preparing Eye Drop Formulation No. 2

Eye drop formulations were prepared as described below. The following products were used in the amounts specified in TABLE 3:

TABLE 3
Pharmaceutical Composition No. 2

| Amount | Product |
|---|---|
| 0.50 g | chondroitin sulfate (bovine) |
| 0.25 g | powdered dextran-70 |
| 0.1 g | edetate disodium powder |
| 0.03 g | potassium chloride |
| 0.20 g | PLURONIC ® F-127 |
| 0.2 g | Glycerol |
| 0.9 g | sodium phosphate dibasic anhydrous |
| 0.18 g | sodium phosphate monobasic anhydrous |
| 100 mL | sterile injectable water |

Chondroitin sulfate, dextran, potassium phosphate, dibasic and monobasic sodium phosphate, potassium chloride, EDTA, and PLURONIC® F-127 were combined with about 90% of water and stirred until completely dissolved, followed by adding glycerol with continued stirring. The pH of the solution was then adjusted to about 7.0-7.4 using sodium hydroxide solution. The solution was then filtered through a 0.2 micron filter into a sterile droptainer and sealed for subsequent topical delivery to the eye as eye drops. The viscosity of this solution is about the same as water.

### Example 3

### Preparing Eye Drop Formulation Nos. 3 and 4

An eye drop formulation was prepared as described below. The following products were used in the amounts specified in TABLE 4:

TABLE 4
Preparation of Pharmaceutical Composition Nos. 3 and 4

| Amount | Product |
|---|---|
| 0.02 or 0.04 g | betamethasone sodium phosphate |
| 0.25 g | chondroitin sulfate (bovine) |
| 0.25 g | powdered dextran-70 |
| 0.1 g | edetate disodium powder |
| 0.10 g | powdered sodium thiosulfate pentahydrate |
| 0.03 g | potassium chloride |
| 0.20 g | PLURONIC ® F-127 |
| 0.2 g | glycerol |
| 0.9 g | sodium phosphate dibasic anhydrous |
| 0.18 g | sodium phosphate monobasic anhydrous |
| 100 mL | sterile injectable water |

Chondroitin sulfate, dextran, sodium thiosulfate, phosphate buffer, potassium chloride, EDTA, and PLURONIC® F-127 were combined with about 90% of the water and stirred until completely dissolved followed by addition of glycerol with continued stirring. The pH of the solution was then adjusted to about 7.0 using sodium hydroxide. A stock solution of 1% betamethasone sodium phosphate was prepared by dissolving 1 gm of betamethasone sodium phosphate powder in 100 ml water and confirming the stock solution concentration by HPLC. Then 2 (Formulation 3) or 4 ml (Formulation 4) of the 1% betamethasone sodium phosphate stock solution was then added to the mixture with continued stirring. The solution was then filtered through a 0.2 micron filter into sterile dropper bottles or unit dose vials for subsequent topical delivery to the eye as eye drops. The viscosities of these solutions are about the same as water.

### Example 4

### Preparing Eye Drop Formulation No. 5

An eye drop formulation was prepared as described below. The following products were used in the amounts specified in TABLE 5:

TABLE 5
Preparation of Pharmaceutical Composition No. 5

| Amount | Product |
|---|---|
| 0.2 g | betamethasone sodium phosphate |
| 0.25 g | chondroitin sulfate (bovine) |
| 0.25 g | powdered dextran-70 |
| 0.1 g | edetate disodium powder |
| 0.10 g | powdered sodium thiosulfate pentahydrate |
| 0.015 g | potassium chloride |
| 0.20 g | PLURONIC ® F-127 |
| 0.2 g | Glycerol |
| 0.95 g | sodium phosphate dibasic anhydrous |
| 0.2 g | sodium phosphate monobasic anhydrous |
| 100 mL | sterile injectable water |

Chondroitin sulfate, dextran, sodium thiosulfate, phosphate buffer, potassium chloride, EDTA, and PLURONIC® F-127 were combined with about 90% of the water and stirred until completely dissolved followed by addition of glycerol with continued stirring. The pH of the solution was then adjusted to about 7.0 using sodium hydroxide. A stock solution of 1% betamethasone sodium phosphate was prepared by dissolving 1 gm of betamethasone sodium phosphate powder in 100 ml water and confirming the stock solution concentration by HPLC. Then 20 ml of the 1% betamethasone sodium phosphate stock solution was then added to the mixture with continued stirring. The solution was then filtered through a 0.2 micron filter into sterile dropper bottles or unit dose vials for subsequent topical delivery to the eye as eye drops. The viscosity of this solution is about the same as water

### Example 5

### Preparing Eye Drop Formulation No. 6

An eye drop formulation was prepared as described below. The following products were used in the amounts specified in TABLE 6:

TABLE 6
Preparation of Pharmaceutical Composition No. 6

| Amount | Product |
|---|---|
| 0.1 g | chondroitin sulfate (bovine) |
| 0.1 g | powdered dextran-70 |
| 0.003 g | potassium chloride |
| 0.20 g | PLURONIC ® F-127 |
| 1.00 g | Glycerol |
| 0.6 g | sodium phosphate dibasic anhydrous |
| 0.1 g | sodium phosphate monobasic anhydrous |
| 100 mL | sterile injectable water |

Chondroitin sulfate, dextran, phosphate buffer, potassium chloride, and, PLURONIC® F-127 were combined with about 90% of the water and stirred until completely dissolved followed by addition of glycerol with continued stirring. The pH of the solution was then adjusted to about 7.0 using sodium hydroxide. The solution was then filtered through a 0.2 micron filter into sterile dropper bottles or unit dose vials for subsequent topical delivery to the eye as eye drops. The viscosity of this solution is about the same as water.

Although the invention has been described with the reference to the above examples, it will be understood that modifications and variations are encompassed within the spirit and scope of the invention. Accordingly, the invention is limited only by the finally granted claims.

What is claimed is:

1. A composition for treating optical pain or discomfort, the composition comprising an eye dropper bottle or an eye dropper dose vial containing an eye drop formulation, the eye drop formulation comprising a viscosity about the same as water and chondroitin sulfate at a concentration of 0.1% to 2.5% wt/wt.

2. The composition of claim 1, wherein the eye drop formulation further comprises dextran-70 and glycerol.

3. The composition of claim 1, wherein the eye drop formulation is free of hyaluronic acid and glucosamine sulfate.

4. The composition of claim 1, wherein the eye drop formulation comprises 0.1% wt/wt chondroitin sulfate, 0.1% wt/wt dextran-70, 0.03% wt/wt potassium chloride, 0.20% wt/wt PLURONIC F-127, 1% wt/wt glycerol, 0.6% wt/wt sodium phosphate dibasic anhydrous, 0.1% wt/wt sodium phosphate monobasic dihydrous and water.

5. A composition for treating optical pain or discomfort, the composition comprising an eye dropper bottle or an eye dropper dose vial containing an eye drop formulation, the eye drop formulation comprising a viscosity of 1 to 25 centipoise (cps) and chondroitin sulfate a concentration of 0.1% to 2.5% wt/wt.

6. The composition of claim 5, wherein the viscosity is 1 to 12 cps.

7. The composition of claim 6, wherein the viscosity is 1 to 5 cps.

8. A method for treating ocular pain or discomfort, the method comprising topically administering to an eye of a subject suffering from ocular pain or discomfort, a therapeutically effective amount of the eye drop formulation using the composition of claim 1.

9. The method of claim 8, wherein the ocular pain or discomfort is from an eye allergy.

10. The method of claim 8, wherein the ocular pain or discomfort is from a corneal abrasion.

11. The method of claim 8, wherein the ocular pain or discomfort is from wearing a contact lens.

12. The method of claim 8, wherein the ocular pain or discomfort is from surgical temporary ocular discomfort syndrome (STODS).

13. The method of claim 8, wherein the ocular pain or discomfort is from ocular surgery.

14. The method of claim 13, wherein the ocular surgery is cataract surgery or refractive surgery.

15. The method of claim 8, wherein the ocular pain or discomfort is from an ocular surface disease.

16. The method of claim 15, wherein the ocular surface disease is selected from the group consisting of dry eye disease, contact lens induced ocular surface dry eye disease, and preservative induced ocular surface disease.

17. The method of claim 8, wherein the eye drop formulation is administered over at least 7 days, optionally for 14 days or more.

18. A method for treating ocular pain or discomfort, the method comprising topically administering to an eye of a subject suffering from ocular pain or discomfort, a therapeutically effective amount of the eye drop formulation using the composition of claim 5.

19. The method of claim 18, wherein the ocular pain or discomfort is from a condition selected from the group consisting of an eye allergy, a corneal abrasion, contact lens wear, STODS, ocular surgery, and an ocular surface disease.

20. The method of claim 19, wherein the ocular surger is cataract surgery or refractive surgery.

21. The method of claim 19, wherein the ocular surface disease is selected from the group consisting of dry eye disease, contact lens induced ocular surface dry eye disease, and preservative induced ocular surface disease.
